(19) United States
(12) Patent Application Publication
(10) Pub. No.: US 2012/0322760 A1
(43) Pub. Date: Dec. 20, 2012

(54) METHODS OF TREATMENT WITH A LOW MOLECULAR WEIGHT HEPARIN COMPOSITION

(75) Inventors: Ian Fier, Southborough, MA (US); James Roach, Sudbury, MA (US); Sunil Rao, Chapel Hill, NC (US); Richard Clinton Becker, Durham, NC (US)

(73) Assignee: MOMENTA PHARMACEUTICALS, INC., Cambridge, MA (US)

(21) Appl. No.: 13/498,111

(22) PCT Filed: Sep. 22, 2010
(86) PCT No.: PCT/US10/49890

§ 371 Date: Sep. 4, 2012

Related U.S. Application Data
(60) Provisional application No. 61/245,089, filed on Sep. 23, 2009.

(51) Int. Cl. A61K 31/727; A61B 5/021; A61B 5/0205; A61B 5/145; A61P 7/04; A61P 9/00

(52) U.S. Cl. 514/56; 600/301; 600/309; 600/485

(57) ABSTRACT

Methods of identifying and selecting subjects for treatment with a low molecular weight heparin (LMWH) composition are provided. Methods of treatment with the LMWH compositions are also provided.

This application claims priority to provisional patent application Ser. No. 61/245,089, filed Sep. 23, 2009. The disclosures of the prior application is considered part of (and is incorporated by reference in) the disclosure of this application.

### BACKGROUND

Coagulation is a physiological pathway involved in maintaining normal blood hemostasis in mammals. Under conditions in which a vascular injury occurs, the coagulation pathway is stimulated to form a blood clot to prevent the loss of blood. Immediately after the vascular injury occurs, blood platelets begin to aggregate at the site of injury forming a physical plug to stop the leakage. In addition, the injured vessel undergoes vasoconstriction to reduce the blood flow to the area and fibrin begins to aggregate forming an insoluble network or clot, which covers the ruptured area.

When an imbalance in the coagulation pathway shifts towards excessive coagulation, the result is the development of thrombotic tendencies, which are often manifested as heart attacks, strokes, deep vein thrombosis, and acute coronary syndromes such as myocardial infarcts, and unstable angina. Furthermore, an embolism can break off from a thrombus and result in a pulmonary embolism or cerebral vascular embolism including stroke or transient ischemia attack. Current therapies for treating disorders associated with imbalances in the coagulation pathway involve many risks and must be carefully controlled.

### SUMMARY OF THE INVENTION

In a Phase 2a trial of M118-REH in elective percutaneous coronary intervention (PCI), it was unexpectedly discovered that M118-REH demonstrated lower bailout glycoprotein (GP) IIb/IIIa utilization than the comparator unfractionated heparin (UFH) at all dosages of M118-REH tested.

Accordingly, in one aspect, the invention features methods of selecting and/or treating patients who have, or are at risk of having, a thrombotic or cardiovascular disorder (e.g., who have acute coronary syndrome, e.g., unstable angina, non-ST segment elevated myocardial infarction (NSTEMI) or a ST segment elevated myocardial infarction (STEMI)). The methods include selecting such a patient for treatment with a low molecular weight heparin composition described herein, e.g., selecting such a patient for treatment with a low molecular weight heparin composition described herein in the absence of treatment with a GPIIb/IIIa inhibitor, and/or treating the patient with low molecular weight heparin composition described herein (e.g., in the absence of treatment with a GPIIb/IIIa inhibitor), where the patient is selected on the basis of having one or more of the following characteristics:

(a) the patient is at risk of a major or minor bleeding event and/or has a predisposition to bleeding (e.g., has exhibited one or more bleeding event within the past 27, 28, 29, 30, 31 or 32 days);

(b) the patient has a platelet count less than a reference standard (e.g., less than 150,000 platelets/mm³) or has had a platelet count less than a reference standard after a previous treatment with a GPIIb/IIIa inhibitor (e.g., abciximab, eptifibatide or tirofiban). In some embodiments, the patient has a platelet count lower than a reference standard by 5%, 10%, 15%, 20%, 25%, 30%, 35%, 40%, 45%, 50%, 55%, 60%, 65%, 70%, 75%, 80%, 85%, or 90%. The method may include a step of evaluating platelet count in the patient.

(c) the patient has creatinine clearance levels less than a reference standard, e.g., less than 50 mL/min using the Cockcroft-Gault equation. In some embodiments, the patient has creatinine clearance levels lower than the reference standard by 5%, 10%, 15%, 20%, 25%, 30%, 35%, 40%, 45%, 50%, 55%, 60%, 65%, 70%, 75%, 80%, 85%, or 90%. The method may include a step of evaluating creatinine clearance levels in the patient.

(d) the patient has a systolic blood pressure level and/or diastolic blood pressure level greater than a standard, e.g., greater than 180 mm Hg, or 110 mm Hg. In some embodiments, the patient has systolic blood pressure level and/or diastolic blood pressure levels greater than the reference standard by 5%, 10%, 15%, 20%, 25%, 30%, 35%, 40%, 45%, 50%, 55%, 60%, 65%, 70%, 75%, 80%, 85%, or 90%. The method may include a step of determining systolic blood pressure level and/or diastolic blood pressure level of the patient. The method may include a step of evaluating systolic blood pressure levels and/or diastolic blood pressure levels.

(e) the patient receives dialysis treatment;

(f) the patient has tested positive for the production of antibodies to a GPIIb/IIIa inhibitor, e.g., abciximab, or is at risk for developing antibodies to treatment with a GPIIb/IIIa inhibitor, e.g., abciximab. The method may include a step of determining if antibodies to a GPIIb/IIIa inhibitor, e.g., abciximab, are present in the patient (e.g., via ELISA or radioimmunoprecipitation assay (RIP);

(g) the patient has previously experienced an infusion site reaction (e.g., during or within 12 hours of infusion) to a GPIIb/IIIa inhibitor, e.g., abciximab, eptifibatide or tirofiban, or is at risk for developing such an infusion site reaction. The method may include the step of determining if the patient has previously experienced an infusion site reaction to a GPIIa/IIIb inhibitor, e.g., a GPIIa/IIIa inhibitor described herein.

In some embodiment, where the patient is selected on the basis of any of (a)-(g), the method further includes administering a low molecular weight heparin composition described herein to the subject, e.g., at a dose between about 60 IU/kg and about 150 IU/kg (e.g., 70 IU/kg, 75 IU/kg, 80 IU/kg, 85 IU/kg, 90 IU/kg, 95 IU/kg. 100 IU/kg, 110 IU/kg, 115 IU/kg, 120 IU/kg or 125 IU/kg). The low molecular weight heparin composition described herein may be administered subcutaneously or by intravenous (i.v.) infusion. In some embodiments, the treatment further includes a surgical intervention such as PCI, stent placement or atherectomy;

In a second aspect, the invention features a method for evaluating a subject having or at risk of having a thrombotic or cardiovascular disorder, e.g., a subject who has been selected to receive treatment with an anticoagulant. The method includes: (a) evaluating the subject for one or more (e.g., 1, 2, 3, 4 or 5 or more) of the following parameters: bleeding risk, platelet count (e.g., current platelet count or platelet count from a previous treatment with a GPIIb/IIIa inhibitor), creatinine clearance levels, infusion site reaction (e.g., infusion site reaction from a previous treatment with a GPIIb/IIIa inhibitor), presence of antibodies to a GPIIb/IIIa inhibitor, or blood pressure; and based on the evaluation making a decision for the subject about treatment with a low molecular weight heparin composition described herein or another anticoagulant (e.g., UFH, enoxaparin, fondaparinux, bivalirudin). In some embodiments, if the subject has a value for one or more of the parameters that differs from the value for a standard (e.g., as described hereinabove), the decision is made to treat the subject with a low molecular weight heparin composition described herein, e.g., instead of another anticoagulant. In some embodiments, a sample is obtained from the subject and the sample is modified, e.g., concentrated, diluted, a reagent is added to the sample, e.g., a detectable reagent, e.g., a reagent which detects the absence or presence antibodies to a GPIIb/IIIa inhibitor.

In some embodiments of the aspects described above, the LMWH composition has: a weight average molecular weight of about 5000 to 9000 Da, e.g., about 5000 to 8300 Da, e.g., about 5500 to 8000 Da, e.g., about 5700 to 7900 Da, e.g., about 5800 to 6800 Da; and

an anti-IIa activity of about 50 to 300, e.g., about 70 to 280, e.g., about 90 to 250 IU/mg, e.g., about 100 to 250 IU/mg, e.g., about 110 to 200 IU/mg, about 100 to 140 IU/mg, about 120 to 200 IU/mg, about 150 to 200 IU/mg, about 130 to 190 IU/mg, e.g., about 155 to 195 IU/mg.

In some embodiments of the aspects described above, the LMWH composition has: a weight average molecular weight of about 5000 to 9000 Da, e.g., about 5000 to 8300 Da, e.g., about 5000 to 8000 Da, about 5500 to 8000 Da, e.g., about 5700 to 7900 Da, e.g., about 5800 to 6800 Da; and

anti-IIa activity that is at least 50%, 60%, 70%, 80%, 85%, 90%, 95%, 99% or 100% neutralizable with protamine, e.g., as measured by activated partial thromboplastin time (ACT) or activated partial thromboplastin time (aPTT). Preferably, the anti-IIa activity of the LMWH is neutralized by at least 50%, 60%, 70%, 80%, 85%, 90%, 95%, 99% or 100% within 5, 10, 15, 30 minutes after protamine administration.

In some embodiments of the aspects described above, the LMWH composition has: a weight average molecular weight of about 5000 to 9000 Da, e.g., about 5000 to 8300 Da, e.g., about 5500 to 8000 Da, e.g., about 5700 to 7900 Da, e.g., about 5800 to 6800 Da; and

ΔUH_{NAc,6S}GH_{NS,3S,6S} is 5 to 15%, e.g., 7 to 14%, e.g., 9 to 12%, of the composition, e.g., as measured by mole %. Preferably the ΔUH_{NAc,6S}GH_{NS,3S,6S} at the non-reducing end of the molecule of about 5 to 15%, e.g., 7 to 14%, e.g., 9 to 12%, of the chains in the composition, e.g., as measured by mole %.

In some embodiments of the aspects described above, the LMWH composition has: an average chain length of about 9 to 18 disaccharides or 8 to 18 disaccharides, e.g., about 9 to 16, 10 to 16 or 8 to 16 disaccharides; and

ΔUH_{NAc,6S}GH_{NS,3S,6S} is 5 to 15%, e.g., 7 to 14%, e.g., 9 to 12%, of the composition, e.g., as measured by mole %. Preferably the ΔUH_{NAc,6S}GH_{NS,3S,6S} at the non-reducing end of the molecule of about 5 to 15%, e.g., 7 to 14%, e.g., 9 to 12%, of the chains in the composition, e.g., as measured by mole %.

In some embodiments of the aspects described above, the LMWH composition has: a weight average molecular weight of 5000 to 9000 Da, e.g., about 5000 to 8300 Da, e.g., about 5500 to 8000 Da, e.g., about 5700 to 7900 Da, e.g., about 5800 to 6800 Da; and

an anti Xa to anti-IIa ratio of 3:1 or less, e.g., 2:1, e.g., 1.6:1, 1.5:1, 1.4:1, 1.3:1, 1.2:1, 1.1:1, 1:1 or 0.5:1.

Preferably, the anti-Xa to anti-IIa ratio remains relatively constant over the course of an administration of the LMWH preparation, e.g., the anti-Xa to anti-IIa ratio varies no more than about ±1.5, ±1, ±0.5, or ±0.2, over a period of about 30, 60, 120, 180, 240, 300 minutes. For example, if an initial ratio of anti-Xa activity to anti-IIa activity is 2, then the ratio measured at a second time (e.g., 30, 60, 120, 180, 240, 300 minutes) after the initial administration will preferably be less than 3, and preferably at or around 2.

In some embodiments of the aspects described above, the LMWH composition has: one or more of the following characteristics:

the composition has substantially no (e.g., at least 85%, 90%, 95% or more of the chains do not have) modified reducing end structures; at least 60%, 70%, 80%, 85%, 90%, 95%, 99% of the chains of the composition have H_{NAc} at the reducing end; less than 90%, 95%, 98%, 99%, preferably none of the chains of the composition have a sulfated ΔU at the non-reducing end; there is substantially no linkage region (e.g., less than 0.1% linkage region) present in the composition; the composition has more chains with 3-O sulfates than commercially available LMWHs, e.g., enoxaparin or dalteparin; and the ratio of ΔUH_{NAc,6S}GH_{NS,3S,6S} to ΔU_{2S}H_{NS,6S}IH_{NAc,6S}GH_{NS,3S,6S} in the composition is about 1:1 to 4:1.

In one embodiment, the composition has two, three, four, five or all of these characteristics.

In some embodiments of the aspects described above, the LMWH composition has the following structure:

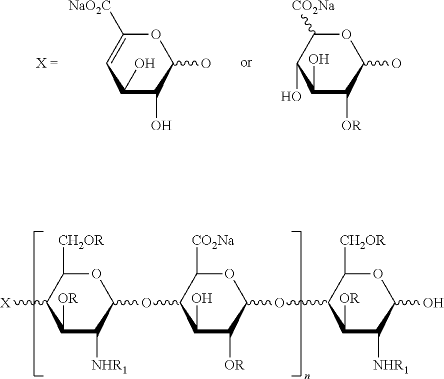

wherein R is H or SO₃X;

R1 is SO₃X or COCH₃;

X is a monovalent or divalent cation;

n=2-50, e.g., 2-40; and

the composition preferably has an average value for n of 9-16, 10-16, 8-16 or 8-15.

In some embodiments of the aspects described above, the LMWH composition includes:

oligosaccharide chains that have the following structure:

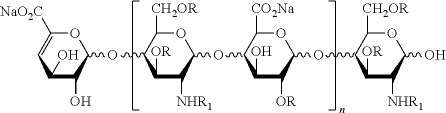

and oligosaccharide chains that have the following structure:

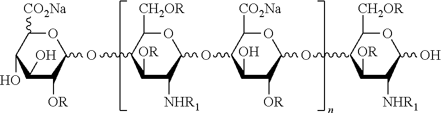

wherein R is H or SO₃X;

R1 is SO₃X or COCH₃;

X is a monovalent or divalent cation;

n=2-50, e.g., 2-40; and

the composition preferably has an average value for n of 9-16, 10-16, 8-16 or 8-15.

In a preferred embodiment, the LMWH composition includes:

oligosaccharide chains that have the following structure:

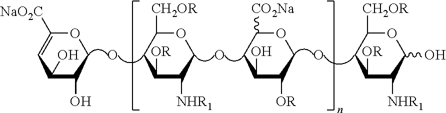

and oligosaccharide chains that have the following structure:

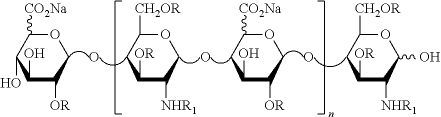

R is H or SO₃Na;

R1 is SO₃Na or COCH₃;

n=2-50, e.g., 2-40; and

the composition preferably has an average value for n of 9 to 16, 10 to 16 or 8 to 15.

Any of the LMWHs described herein, e.g., described above, can have other properties. E.g., one of the above described compositions can further have one or more of functional or structural properties set out below.

Thus, in one embodiment, the LMWH composition has an anti-Xa activity of about 100 to 400 IU/mg, e.g., about 120 to 380 IU/mg, e.g., about 150 to 350 IU/mg, e.g., about 160 to 330 IU/mg, about 170 to 330 IU/mg, e.g., about 180 to 300 IU/mg, e.g., about 150 to 200 IU/mg, 200 to 300 IU/mg, 130 to 220 IU/mg, 225 to 274 IU/mg.

In one embodiment, the LMWH composition has an anti-Xa activity that is at least 50%, 60%, 70%, 80%, 85%, 90%, 95%, 99%, 100% neutralizable, e.g., as measured by anti-Xa activity, ACT or aPTT. Preferably, the anti-Xa activity is neutralized by at least 50%, 60%, 70%, 80%, 85%, 90%, 95%, 99% or 100% within 5, 10, 15 minutes after protamine administration. For example, the anti-Xa activity can be neutralized by at least 50%, 60%, 70%, 80%, 85%, 90%, 95%, 99% or 100% within 5, 10, 15, 30 minutes after protamine administration at a dose of about 1, 2, 3 mg of the LMWH composition per 100 anti-Xa IU of plasma.

In another embodiment, the LMWH composition has one or more of the following properties: the activity of the composition can be monitored by aPTT and/or ACT; the polydispersity of the composition is less than 1.6, e.g., the polydispersity is about 1.6 to 1.1, e.g., 1.5 to 1.1, e.g., 1.4 to 1.1, e.g., 1.3 to 1.1, e.g., 1.2 to 1.1; less than 70%, 60%, 50%, 45%, 40%, 35%, 30% of the chains present in the composition have a molecular weight greater than 7500 or 8000 Da; less than 40%, 35%, 30%, 25% of the chains present in the composition have a molecular weight less than 5500 or 5000 Da; the composition comprises a mixture of ΔU and I/G structures at the non-reducing ends of the chains; and fewer chains in the composition have PF4 binding sites than enoxaparin, dalteparin, UFH.

In one embodiment, about 15%, 20%, 25%, 30%, 35%, 45%, 50% of the chains in the LMWH composition have a ΔU at the non-reducing end. Preferably, about 15% to 50%, e.g., 15% to 35% of the chains, e.g., 20% to 35% of the chains in the composition have a ΔU at the non-reducing end.

In one embodiment, the LMWH composition has a higher degree of sulfation than enoxaparin or dalteparin. In one embodiment, the LMWH composition has more trisulfated disaccharides present in the composition than enoxaparin or dalteparin, e.g., the LMWH composition has about 50 to 65% trisulfated disaccharides, e.g., 55 to 60%, 55 to 58%, 57 to 60% trisulfated disaccharides, as determined by mole %.

In one embodiment, the composition comprises a higher level of ΔUH_{NAc,6S}GH_{NS,3S,6S} than enoxaparin, daltaparin and/or UFH, e.g., comprises about 5 to 15 mole %, e.g., 7 to 14 mole %, e.g., 9 to 12 mole %.

In one embodiment, the LMWH composition has a calcium content less than 3%, 2.5%, 2%, 1.5%, 1.0%, and/or a sodium content less than 30%, 25%, 20%, 15%, 10%. In one embodiment, the LMWH composition comprises: less than 1000 ng/mg, 750 ng/mg, 500 ng/mg, 250 ng/mg of a heparinase enzyme, e.g., a heparinase enzyme described herein; less than 1.0%, 0.5%, 0.3% w/w methanol; less than 1.0%, 0.5%, 0.3%, 0.1% w/w ethanol; less than 2.0%, 1.75%, 1.25%, 1.0%, 0.5%, 0.3%, 0.15% chloride; less than 15%, 10%, 5%, 2.5% water by weight; less than 2000, 1500, 1000, 950, 900, 850, 800, 750, 700, 650, 600, 550, 500, 450, 400, 350, 300 ppm of free sulfate.

In one embodiment, the LMWH composition provides increased TFPI release as compared to enoxaparin. In one embodiment, the LMWH provides at least a 2, 3, 4, 5, 6, 7, 8, 9, 10, 15, 20, 30, 40 fold increase in TFPI release as compared to enoxaparin.

In one embodiment, the LMWH composition has an intravenous half life of about 30 minutes to 3 hours, e.g., about 1 to 2 hours. In one embodiment, the LMWH composition has a subcutaneous half life of about 30 minutes to 3.0 or 3.5 hours, e.g., about 1.5 to 2.5 hours, e.g., about 2 hours.

In one embodiment, the LMWH composition has one or more of the following characteristics:

the composition has substantially no (e.g., at least 85%, 90%, 95% or more of the chains do not have) modified reducing end structures; at least 60%, 70%, 80%, 85%, 90%, 95%, 99% of the chains of the composition have H_{NAc} at the reducing end; less than 90%, 95%, 98%, 99%, preferably none of the chains of the composition have a sulfated ΔU at the non-reducing end; there is substantially no linkage region (e.g., less than 0.1% linkage region) present in the composition; the composition has more chains with 3-O sulfates than commercially available LMWHs, e.g., enoxaparin or dalteparin; and the ratio of ΔUH_{NAc,6S}GH_{NS,3S,6S} to ΔU_{2S}H_{NS,6S}IH_{NAc,6S}GH_{NS,3S,6S} in the composition is about 1:1 to 4:1 (e.g., 1:1. 2:1, 3:1 or 4:1). In one embodiment, the LMWH composition has two, three, four, five or all of these characteristics.

In another embodiment of the aspects described above, the LMWH composition has the following properties:
a weight average molecular weight of about 5000 to 9000 Da;
anti-IIa activity of about 50 to 300 IU/mg, e.g., 100 to 250 IU/mg, e.g., 110 to 250 IU/mg or 120 to 250 IU/mg;
anti-IIa activity that is at least 50% neutralizable with protamine, e.g., as measured by ACT or aPTT;
ΔUH_{NAc,6S}GH_{NS,3S,6S} is 5 to 15% of the composition, preferably ΔUH_{NAc,6S}GH_{NS,3S,6S} at the non-reducing end of about 5 to 15% of the composition;
an average chain length of about 9 to 16 disaccharides;
an anti Xa to anti-IIa ratio of 3:1 or less;
the anti-Xa to anti-IIa ratio remains relatively constant over the course of an administration of the LMWH, e.g., the anti-Xa to anti-IIa ratio varies no more than about ±1.5, ±1, ±0.5, or ±0.2, over a period of about 30, 60, 120, 180, 240, 300 minutes. For example, if an initial ratio of anti-Xa activity to anti-IIa activity is 2, then the ratio measured at a second time (e.g., 30, 60, 120, 180, 240, 300 minutes) after the initial administration will preferably be less than 3, and preferably at or around 2.

In a preferred embodiment, the LMWH composition has the following properties:

anti-Xa activity of about 100 to 400 IU/mg, e.g., 150 to 350 IU/mg, 160 to 350 IU/mg or 180 to 330 IU/mg;

anti-Xa activity that is at least 50% neutralizable, e.g., as measured by anti-Xa activity, ACT or aPTT;

a polydispersity of less than 1.6;

less than 70%, 60%, 50% of the chains present in the composition have a molecular weight greater than 7500 Da;

less than 40% of the chains present in the composition have a molecular weight less than 5000 Da;

it includes a mixture of ΔU and I/G structures at the non-reducing ends of the chains;

it has substantially no modified reducing end structures;

fewer chains in the composition have PF4 binding sites than enoxaparin, dalteparin, or UFH;

at least 60%, 70%, 80%, 90% of the chains of the composition have HNAc at the reducing end;

about 15% to 35% of the chains in the composition have a ΔU at the non-reducing end;

less than 90%, 95%, 98%, 99%, preferably none of the chains of the composition have a sulfated ΔU at the non-reducing end.

In a preferred embodiment, the LMWH composition has the following properties:

it has a higher degree of sulfation than enoxaparin or dalteparin;

it has more trisulfated disaccharides present in the composition than enoxaparin or dalteparin, e.g., the LMWH composition has about 50 to 65% trisulfated disaccharides, as determined by mole %

it has a higher level of ΔUH_{NAc,6S}GH_{Ns,3S,6S} than enoxaparin, daltaparin and/or UFH, e.g., ΔUH_{NAc,6S}GH_{NS,3S,6S} is present at about 5 to 15 mole %.

In a preferred embodiment, the LMWH composition has the following properties:

it has a calcium content less than 3% and/or a sodium content less than 20%;

it includes less than 1000 ng/mg of a heparinase enzyme;

it has less than 1.0% w/w methanol;

it has less than 1.0% w/w ethanol;

it has less than 2.0% chloride;

it has less than 15% water by weight;

it has less than 2000 ppm of free sulfate.

In a preferred embodiment, the LMWH composition has the following properties:

it provides increased tissue factor pathway inhibitor (TFPI) release as compared to enoxaparin.

In a preferred embodiment, the LMWH composition has an intravenous half life of about 30 minutes to 3 hours.

In a preferred embodiment, the LMWH composition includes:

oligosaccharide chains that have the following structure:

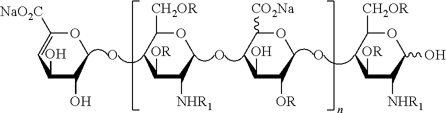

and oligosaccharide chains that have the following structure:

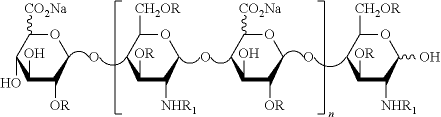

wherein

R is H or SO₃Na;

R₁ is SO₃Na or COCH₃;

n=2-50;

wherein the composition has the following properties:
(a) an average value for n of 9-16,
(b) 5 to 15% ΔUH_{NAc,6S}GH_{NS,3S,6S} as measured by mole %,
(c) weight average molecular weight of 5500 to 8500 Da,
(d) anti-Xa activity of 120 to 380 IU/mg, e.g., 160 to 350 IU/mg or 170 to 330 IU/mg,
(e) an anti-Xa to anti-IIa ratio of 2:1 to 1:1, and
(f) the ratio of (e) is constant over a period of about 30 to 120 minutes after administration to a subject.

In another embodiment of the aspects described above, the LMWH composition is a pharmaceutical composition described herein.

In some embodiments of the aspects described above, the subject has, or at risk of having, a disorder or condition selected from the group consisting of: a disorder associated with thrombosis or cardiovascular disease, e.g., acute coronary syndrome (ACS), stable or unstable angina, myocardial infarction, e.g., ST-segment elevated myocardial infarction (STEMI) or non-ST-segment elevated myocardial infarction (NSTEMI), vascular conditions or atrial fibrillation. The subject can be undergoing, or have undergone, a surgical procedure, e.g., percutaneous coronary intervention (PCI), stent placement, angioplasty, or coronary artery bypass graft surgery (CABG). The compositions of the invention are administered to a subject having or at risk of developing one or more of the diseases in an effective amount for treating the disorder or condition.

In one embodiment, the method further includes monitoring the activity of the LMWH composition in the subject using a coagulation assay, e.g., using ACT and/or aPTT.

In one embodiment, the method further includes administering protamine sulfate after administration of the LMWH composition to neutralize some or all of the activity, e.g., anti-Xa activity and/or anti-IIa activity, of the LMWH composition. In one embodiment, about 50%, 60%, 70%, 80%, 90%, 95% or all of the anti-IIa activity of the LMWH composition is neutralized, e.g., about 50%, 60%, 70%, 80%, 90%, 95% or all of the anti-IIa activity of the LMWH composition is neutralized within 5, 10, 15, 20, 25, 30, 40 minutes after protamine administration. In one embodiment, about 50%, 60%, 70%, 80%, 90%, 95% or all of the anti-IIa activity of the LMWH composition is neutralized, e.g., about 50%, 60%, 70%, 80%, 90%, 95% or all of the anti-IIa activity of the LMWH composition is neutralized within 5, 10, 15, 20, 25, 30, 40 minutes after protamine administration. In one embodiment, protamine sulfate is administered at a dose of about 1 mg, 2 mg, 3 mg, 5 mg of the LMWH composition per 100 anti-Xa IU of plasma. Neutralization of anti-Xa activity and/or anti-IIa activity can be determined, e.g., by ACT and/or aPTT.

In an embodiment of the aspects described above, the disorder is one or more of ACS, myocardial infarction, e.g., NSTEMI OR STEMI, stable angina and unstable angina. Preferably, the thrombotic disorder is arterial thrombosis, e.g., including STEMI. The disorder can be, e.g., associated with surgical intervention, e.g., PCI, stent placement or angioplasty. For example, the subject can have, or be at risk of having, or be recovering from, a surgical intervention, e.g., cardiology intervention (e.g., angioplasty, PCI, stent placement). In one embodiment, the subject is at risk for (e.g., is being considered for) receiving surgical intervention, e.g., CABG.

In one embodiment, the LMWH composition is administered to the subject intravenously, e.g., at 60 IU/kg and about 150 IU/kg (e.g., 70 IU/kg, 75 IU/kg, 80 IU/kg, 85 IU/kg, 90 IU/kg, 95 IU/kg. 100 IU/kg, 110 IU/kg, 115 IU/kg, 120 IU/kg or 125 IU/kg).

In one embodiment, the method further includes monitoring the activity of the LMWH composition in the subject using a coagulation assay, e.g., using ACT and/or aPTT. In one embodiment, anti-Xa activity and/or anti-IIa activity is monitored, e.g., with ACT and/or aPTT, prior to, during, or after surgical intervention, e.g., angioplasty, PCI, stent placement. In one embodiment, anti-Xa activity and/or anti-IIa activity is monitored, e.g., with ACT and/or aPTT, prior to, during, and/or after administration of the LMWH composition. In some embodiments, anti-Xa activity and/or anti-IIa activity is monitored by ACT, and the dose of LMWH is administered to achieve an ACT of about 200 to 350 seconds.

In one embodiment, the method further includes administering protamine sulfate after administration of the LMWH composition to neutralize some or all of the activity, e.g., anti-Xa activity and/or anti-IIa activity, of the LMWH composition. In one embodiment, at least about 50%, 60%, 70%, 80%, 90%, 95% or all of the anti-IIa activity of the LMWH composition is neutralized, e.g., at least about 50%, 60%, 70%, 80%, 90%, 95% or all of the anti-IIa activity of the LMWH composition is neutralized within 5, 10, 15, 20, 25, 30, 40 minutes after protamine administration. In one embodiment, at least about 50%, 60%, 70%, 80%, 90%, 95% or all of the anti-IIa activity of the LMWH composition is neutralized, e.g., at least about 50%, 60%, 70%, 80%, 90%, 95% or all of the anti-IIa activity of the LMWH composition is neutralized within 5, 10, 15, 20, 25, 30, 40 minutes after protamine administration. In one embodiment, protamine sulfate is administered at a dose of about 1-5 mg, e.g., 1 mg, 2 mg, 3 mg, 5 mg of the LMWH composition per 100 anti-Xa IU of plasma. Neutralization of anti-Xa activity and/or anti-IIa activity can be determined, e.g., by ACT and/or aPTT. In one embodiment, anti-Xa activity and/or anti-IIa activity can be determined, e.g., by ACT and/or aPTT, prior to, during and/or after administration of protamine sulfate. In one embodiment, anti-Xa activity and/or anti-IIa activity is neutralized prior to, during or after surgical intervention. For example, in one embodiment, anti-Xa activity and/or anti-IIa activity can be neutralized during or after a surgical intervention such as angioplasty or PCI. In another embodiment, the LMWH composition is neutralized prior to surgical intervention such as CABG.

In one embodiment, the method further includes monitoring the patient for a negative reaction, e.g., epidural or spinal hematoma, hemorrhage or bleeding.

In one embodiment, the LMWH composition is administered intravenously or subcutaneously.

In another aspect, the invention features, a method of advising on, or providing instructions (e.g., written, oral, or computer generated instructions) for, the use of a LMWH composition described herein. The method includes providing instruction regarding use in the absence of a GPIIb/IIIa inhibitor and/or for use for a subject having one or more of the characteristics described herein. The instructions can also include information regarding the use, e.g., with: patients having abnormal renal function or diabetes or clot bound thrombin; patients who are candidates for PCI, stent placement, CABG, angioplasty, etc.; interventional cardiology patients; patients in need of neutralization of previously administered LMWH, e.g., neutralizing with protamine sulfate; patients at risk of epidural or spinal hematoma, hemorrhage and/or bleeding; patients having a low platelet count (e.g., current platelet count or platelet count from a previous treatment with a GPIIb/IIIa inhibitor); patients that have or have previously had an infusion site reaction (e.g., infusion site reaction from a previous treatment with a GPIIb/IIIa inhibitor); and/or patients having antibodies to a GPIIb/IIIa inhibitor. In one embodiment, the instruction pertains to administration of the LMWH composition for ACS, myocardial infarction, e.g., NSTEMI OR STEMI, stable angina and unstable angina, e.g., administration in a sub population of patients such as patients having abnormal renal function, or elderly patients (e.g., patients over 60 years of age). In one embodiment, the instruction pertains to administration of the LMWH composition for thrombotic disorders, e.g., thrombotic disorders associated with surgical intervention, e.g., PCI, stent replacement or angioplasty.

Other features and advantages of the invention will be apparent from the following detailed description, and from the claims.

### DESCRIPTION OF THE DRAWINGS

The drawings are first briefly described.

FIG. 1 is a schematic of the EMINENCE trial design.

FIG. 2 is a graft depicting the incidence of serious and non-serious adverse events for all treatment groups, unfractionated heparin (UFH) at 70 IU/kg, M118-REH at 50 IU/kg, 75 IU/kg and 100 IU/kg.

FIG. 3 is a graft depicting the incident of bailout with a GPIIb/IIIa inhibitor during treatment with unfractionated heparin (UFH) at 70 IU/kg, M118-REH at 50 IU/kg, 75 IU/kg and 100 IU/kg.

### DETAILED DESCRIPTION

### Optimized LMWHs

In many clinical settings, commercially available LMWH preparations are preferred over UFH preparations because LMWHs have more predictable pharmacokinetics and can be administered subcutaneously. However, currently available LMWH preparations lack many of the desirable properties of UFH such as substantial anti-IIa activity, reversibility (or neutralizability) with protamine sulfate and monitorability. Thus, there are clinical settings where LMWHs are not an optimal or practical treatment choice. The invention features LMWH preparations designed to have properties that are clinically advantageous, e.g., over other commercially available LMWH preparations and UFH preparations. Such properties include, e.g., one or more of: reversibility with proteomine sulfate; predictable or otherwise improved pharmacokinetics, improved anti-IIa activity, as compared, e.g., to enoxaparin; substantially constant anti-Xa activity to anti-IIa activity ratio, e.g., over a period of about 30 to 180 minutes; monitorable activity levels by standard tests such as, e.g., ACT or aPTT; subcutaneous bioavailability; and reduced likelihood to cause occurrence of HIT. LMWHs disclosed herein can also have structural characteristics that distinguish them from other commercially available LMWHs. For example, a LMWH preparation provided herein can have one or more of the following characteristics: substantially undetectable linkage region; an increased amount of 3-O sulfates as compared to commercially available LMWH preparations; a subset of the chains have an unsulfated ΔU at the non-reducing end; a subset of the chains, e.g., a majority, e.g., substantially all of the chains, have an N-acetylated hexosamine at the reducing end; a ratio of ΔUH_{NAc,6S}GH_{NS,3S,6S} to ΔU_{2S}H_{NS,6S}IH_{NAc,6S}GH_{NS,3S,6S} of about 1:1 to 4:1 (e.g., about 1:1, 2:1, 3:1, 4:1), and substantially no modified reducing end structures.

Anti-IIa Activity

LMWH preparations are disclosed herein that include a significant number of chains of sufficient length (which can be described, e.g., in terms of average chain length of the preparation and/or weight average molecular weight of the preparation) to provide anti-IIa activity, e.g., anti-IIa activity of about 50 to 300 IU/mg, about 70 to 280 IU/mg, about 90 to 250 IU/mg, about 100 to 140 IU/mg, about 100 to 140 IU/mg, about 110 to 200 IU/mg, about 120 to 200 IU/mg, 120 about 150 to about 200 IU/mg, about 130 to 190 IU/mg, about 155 to 195 IU/mg. Anti-IIa activity is calculated in International Units of anti-IIa activity per milligram using the statistical methods for parallel line assays. The anti-IIa activity levels described herein are measured using the following principle.

M118+ATIII→[M118ATIII]

IIa

M118ATIII→[M118ATIIIIIa]+IIa(Excess)

IIa(Excess)+Substrate→Peptide+pNA(measured spectrophotometrically)

Anti-factor IIa activity is determined by the sample potentiating effect on antithrombin (ATIII) in the inhibition of thrombin. Thrombin excess can be indirectly spectrophotometrically measured. The anti-factor IIa activity can be measured, e.g., on a Diagnostica Stago analyzer or on an ACL Futura3 Coagulation system, with reagents from Chromogenix (S-2238 substrate, Thrombin (53nkat/vial), and Antithrombin), or on any equivalent system. Analyzer response is calibrated using the 2nd International Standard for Low Molecular Weight Heparin.

Chain Length/Molecular Weight

A determination of whether a LMWH preparation includes chains of sufficient chain length can be made, for example, by determining the average chain length of the chains in the LMWH preparation and/or by determining the weight average molecular weight of chains within the LMWH preparation. When average chain length is determined, an average chain length of about 5 to 20, e.g., 7 to 18, preferably about 9 to 16 or 8 to 14 disaccharide repeats, indicates that a significant number of chains in the LMWH preparation are of sufficient chain length.

“Average chain length” as used herein refers to the average chain length of uronic acid/hexosamine disaccharide repeats that occur within a chain. The presence of non-uronic acid and/or non-hexosamine building blocks (e.g., attached PEG moieties) is not included in determining the average chain length. Average chain length is determined by dividing the number average molecular weight (Mn) by the number average molecular weight for a disaccharide (500 Da). Methods of determining number average molecular weight are described below using SEC MALS.

Examples of such LMWH preparations include the following:

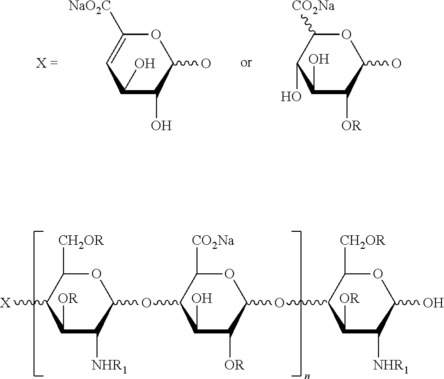

wherein R is H or SO₃X; R1 is SO₃X or COCH₃ and X is a monovalent or divalent cation (e.g., Na or Ca); and average n is about 9 to 16, 10 to 16 or 8 to 15; and

oligosaccharide chains that have the following structure:

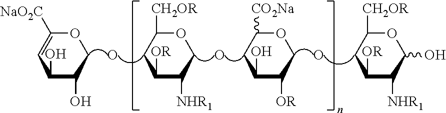

and oligosaccharide chains that have the following structure:

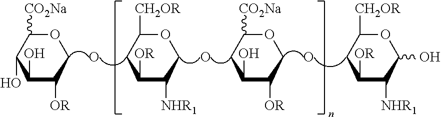

R is H or SO₃X; R1 is SO₃X or COCH₃, X is a monovalent or divalent cation (e.g., Na or Ca); and average n is about 9 to 16, 10 to 16 or 8 to 15.

When weight average molecular weight of a preparation is determined, a weight average molecular weight of about 5000 to 9000 Da, about 5000 to 8300 Da, preferably about 5500 to 8000 Da, about 5700 to 7900, or about 5800 to 6800 Da, indicates that a significant number of chains in the LMWH preparation are of sufficient chain length.

“Weight average molecular weight” as used herein refers to the weight average in daltons of chains of uronic acid/hexosamine disaccharide repeats. The presence of non-uronic acid and/or non-hexosamine building blocks are not included in determining the weight average molecular weight. Thus, the molecular weight of non-uronic acid and non-hexosamine building blocks within a chain or chains in the preparation should not be included in determining the weight average molecular weight. The weight average molecular weight (M_{w}) is calculated from the following equation: M_{w}=Σ(cᵢmᵢ)/Σcᵢ. The variable ci is the concentration of the polymer in slice i and Mi is the molecular weight of the polymer in slice i. The summations are taken over a chromatographic peak, which contains many slices of data. A slice of data can be pictured as a vertical line on a plot of chromatographic peak versus time. The elution peak can therefore be divided into many slices. The weight average molecular weight calculation is average dependant on the summation of all slices of the concentration and molecular weight. The weight average molar weight can be measured, e.g., using the Wyatt Astra software or any appropriate software. The weight average molecular weights described herein are determined by high liquid chromatography with two columns in series, for example a TSK G3000 SWXL and a G2000 SWXL, coupled with a multi angle light scattering (MALS) detector and a refractometric detector in series. The eluent used is a 0.2 sodium sulfate, pH 5.0, and a flow rate of 0.5 mL/min.

Non-Reducing End Structure

In addition to chain length about 5 to 15 mole %, 7 to 14 mole %, or 9 to 12 mole % of the chains in a preparation can have ΔUH_{NAc,6S}GH_{NS,3S,6S} at, or within about two, four or six monosaccharides from the non-reducing end of the chain. Methods that can be used to quantify this structure include, e.g., capillary electrophoresis (CE) and high performance liquid chromatography (HPLC), e.g., reverse phase high performance liquid chromatography (RPHPLC). To quantify the mole % of ΔUH_{NAc,6S}GH_{NS,3S,6S} in a LMWH preparation, a response factor (RF) for ΔUH_{NAc,6S}GH_{NS,3S,6S} can be determined. The determination can also include determining the RF for all species obtained, e.g., using CE or HPLC, e.g., a CE method described herein. To obtain the RF for a species or all species obtained by CE, e.g., a CE method described herein, known concentrations of a standard for the specie or one or more of the species can be injected on the CE and used to determine a RF for each. The RF can then be used to determine the mole %. As described herein, the sample has an actual level of a structure, which can be expressed, e.g., as 5 to 15 when described in units of mole %. That actual level can also be expressed in other units, e.g., weight %. That actual level is the same regardless of the units in which it is expressed. The specification of mole % in the method is merely to indicate the actual prevalence of the structure. The level of structure can be measured in terms of other units and the reference value can be expressed in terms of other units, as long as the reference value as expressed in terms of alternative units corresponds to the same amount of structure as the reference value expressed in mole %, 5 to 15 mole % in this example. Thus, a method which requires showing the structure is present at 5 to 15 mole % can be performed by showing that the structure is present in a range expressed in an alternative unit of measure, e.g., weight %, chain number, or % AUC, wherein the range, as described in the alternative unit of measure, corresponds to the same amount of the structure which would give the mole % referred to, in this example 5 to 15 mole %.

A LMWH preparation described herein can have a mixture of ΔU and iduronic acid (I)/glucuronic acid (G) at the non-reducing end of the chains in the preparation. The nomenclature “ΔU” refers to an unsaturated uronic acid (iduronic acid (I), glucuronic acid (G) or galacturonic acid) that has a double bond introduced at the 4-5 position as a result, e.g., of the lyase action of a heparinase, a HSGAG lyase, other enzyme having similar substrate specificity, or other method of deploymerization. Preferably, about 15% to 35%, 20 to 30% (e.g., 15%, 20%, 25%, 30%, 35%) of the total number of chains in the preparation have a ΔU at the non-reducing end of the chain. The quantity of ΔU and/or I/G at the non-reducing end of chains within the sample can be determined using, e.g., 2D-NMR. In such methods, the total number of chains having an acetylated hexosamine (H_{NAc}) at the reducing end and/or the number of open ring confirmations at the reducing end can be used to determine the total number of chains within the preparation. The total percentage of chains having a ΔU and/or I/G at the non-reducing end can be compared to the total number of chains in the preparation. Preferably, in the LMWH preparations described herein, less than 90%, 95%, 98%, 99% or none of the chains in the preparation have a sulfated ΔU at the non-reducing end.

Reducing End Structures

In some instances, a LMWH preparation provided herein has substantially no modified reducing end structures. In preferred embodiments at least 85%, 90%, 95%, 98%, 99% or all of the chains in the LMWH preparation have a non-modified reducing end structure.

A “modified reducing end structure” refers to a structure that arises at the reducing end of chains in the preparation due to the process of isolating or preparing the preparation from natural sources. For example, many commercially available LMWH preparations are derived from unfractionated heparin primarily through chemical and/or enzymatic depolymerization of the polysaccharide chains. A process used to make a LMWH can cause one or more unique structural modifications to the reducing end of polysaccharide chains of starting material from a natural source. For example, nitrous acid depolymerization of heparin results in the formation of a 2,5-anhydromannose at the reducing end, which can be reduced to form an alcohol, and depolymerization through esterification of the carboxylate functional group on the uronic acid followed by β-elimination results in the formation of a 1,6-anhydro structures at the reducing end of some chains. Thus, 2,5-anhydromannose and 1,6 anhydro structures are examples of modified reduce end structures that can be found on some chains of LMWHs. The chains in a LMWH preparation provided herein can include, e.g., at least about 60%, 70%, 80%, 85%, 90%, 95%, 98%, 99% or all of the chains having an acetylated hexosamine at the reducing end.

Anti-Xa Activity

Anti-Xa activity of a LMWH preparation plays a role in biological activity of LMWH preparations. Preferably, a LMWH preparation provided herein has an anti-Xa activity of about 100 to 400 IU/mg, e.g., about 120 to 380 IU/mg, e.g., about 150 to 350 IU/mg, e.g., about 160 to 350 IU/mg, about 170 to 330 IU/mg, e.g., about 180 to 300 IU/mg, e.g., about 150 to 200 IU/mg, 200 to 300 IU/mg. Anti-Xa activity of a LMWH preparation is calculated in International Units of anti-factor Xa activity per milligram using the statistical methods for parallel line assays. The anti-factor Xa activity of LMWH preparations described herein is measured using the following principle:

M118+ATIII→[M118ATIII]

FXa

M118ATIII→[M118ATIIIFXa]+FXa(Excess)

FXa(Excess)+Substrate→Peptide+pNA(measured spectrophotometrically)

The anti-factor Xa activity is determined by the sample potentiating effect on antithrombin (ATIII) in the inhibition of activated Factor Xa (FXa). Factor Xa excess can be indirectly spectrophotometrically measured. Anti-factor Xa activity can be measured, e.g., on a Diagnostica Stago analyzer with the Stachrom® Heparin Test kit, on an ACL Futura3 Coagulation system with the Coatest® Heparin Kit from Chromogenix, or on any equivalent system. Analyzer response can be calibrated using the NIBSC International Standard for Low Molecular Weight Heparin.

Anti-Xa/IIa Ratio

In some aspects, LMWH preparations provided herein have an anti-Xa activity to anti-IIa activity ratio of 3:1 or less, e.g., 2.1, 1.6:1, 1.5:1, 1.4:1, 1.3:1, 1.2:1, 1.1:1, 1:1. Methods of determining anti-factor Xa activity and the anti-factor IIa activity have been described above. The ratio of anti-factor Xa activity to anti-factor IIa activity is calculated by dividing anti-factor Xa activity (dry basis) by the anti-factor IIa activity (dry basis).

Both anti-Xa activity and anti-IIa activity of heparin and LMWH preparations involve binding of antithrombin III (ATIII) to a specific sequence, represented by the structure ΔUH_{NAc,6S}GH_{NS,3S,6S}, within chains present in the preparation. Binding of ATIII to this sequence mediates anti-Xa activity. In addition, thrombin (factor IIa) binds heparins at a site proximate to the ATIII binding site. Unlike anti-Xa activity that requires only the ATIII binding site, anti-IIa activity requires the presence of an ATIII binding site as well as a chain of sufficient length distal to the ATIII binding site. The anti-IIa activity of LMWH preparations provided herein can be attributed, at least in part, to the presence of ΔUH_{NAc,6S}GH_{NS,3S,6S} at or near the non-reducing end of chains within the LMWH preparations as well as the length of many of the chains present in the preparation. This combination may result in chains within the preparation that contribute to both anti-Xa activity and anti-IIa activity. When both anti-Xa activity and anti-IIa activity are provided by the same chain or chains, the clearance of that chain or chains can result in both a decrease in anti-Xa activity and anti-IIa activity. As such, the anti-Xa activity and anti-IIa activity can remain relatively constant over the course of administration. Therefore, in some aspects, the LMWH preparations provided herein have an anti-Xa activity to anti-IIa activity remains relatively constant over the course of an administration of LMWH, e.g., the anti-Xa activity to anti-IIa activity ratio varies about ±1.5, ±1, ±0.5, or ±0.2, over a period of about 30, 60, 120, 180, 240, 300 minutes. For example, if an initial ratio of anti-Xa activity t anti-IIa activity is 2, then the ratio measured at a second time (e.g., 30, 60, 120, 180, 240, 300 minutes) after the initial administration will preferably be less than 3, and preferably at or around 2.

Neutralization

LMWH preparations provided herein can be neutralized by protamine sulfate. For example, anti-IIa activity and/or anti-Xa activity can be neutralized by at least 50%, 60%, 70%, 80%, 85%, 90%, 95%, 99% or 100% by administration of protamine. Protamine sulfate is commercially available, e.g., from Eli Lilly and Company. Neutralization of anti-Xa activity and anti-IIa activity can be measured, e.g., by standard coagulation assays such as ACT and aPTT, both of which are described further herein. Protamine sulfate can be administered intravenously, e.g., at a dose of about 1, 2, 3 mg per 100 anti-Xa IU of the LMWH preparation in plasma. Preferably, protamine neutralization of anti-Xa activity and/or anti-IIa activity occurs within 5, 10, 15, 20, 25, or 30 minutes after administration of the protamine sulfate.

Polydispersity

The polydispersity of LMWH preparations provided herein is about 1.6 or less, e.g., about 1.6 or 1.5 to 1.1, and numbers in between.

The term “polydisperse” or “polydispersity” refers to the weight average molecular weight of a composition (Mw) divided by the number average molecular weight (Mn). The number average molecular weight (Mn) is calculated from the following equation: Mn=Σci/(Σci/mi). The variable ci is the concentration of the polysaccharide in slice i and Mi is the molecular weight of the polysaccharide in slice i. The summations are taken over a chromatographic peak, which contains many slices of data. A slice of data can be pictured as a vertical line on a plot of chromatographic peak versus time. The elution peak can therefore be divided into many slices. The number average molecular weight is a calculation dependent on the molecular weight and concentration at each slice of data. Methods of determining weight average molecular weight are described above, and were used to determine polydispersity as well.

For any of the ranges described herein, e.g., for a given structure or activity, the ranges can be those ranges disclosed as well as other ranges. For example, a range constructed from a lower endpoint of one range, e.g., for a given building block or activity, can be combined with the upper endpoint of another range, e.g., for the given building block or activity, to give a range.

An “isolated” or “purified” LMWH preparation is substantially free of cellular material or other contaminating proteins from the cell or tissue source from which the LMWH is derived, or substantially free from chemical precursors or other chemicals when chemically synthesized. “Substantially free” means that a preparation of LMWH is at least 50% pure (wt/wt). In a preferred embodiment, the preparation of LMWH has less than about 30%, 20%, 10% and more preferably 5% (by dry weight), of non-heparin polysaccharides, proteins or chemical precursors or other chemicals, e.g., from manufacture. These also referred to herein as “contaminants”. Examples of contaminants that can be present in a LMWH preparation provided herein include, but are not limited to, calcium, sodium, heparinase enzyme (or other enzyme having similar substrate specificity), methanol, ethanol, chloride, sulfate, dermatan sulfate, and chondrotin sulfate.

Methods of Monitoring Activity of a LMWH Preparation

The activity of a LMWH preparation provided herein can be monitored by standard anti-coagulation assays. Such assays include, e.g., ACT and aPTT, both of which are routinely practiced in hospitals and specifically hospital operating rooms.

ACT is a test that is used to monitor the effectiveness of heparin therapy. The ACT can be done at the bedside, e.g., for patients experiencing pulmonary embolus, extracorporeal membrane oxygenation (ECMO) and hemodialysis. ACT is most often used before, during and after surgical intervention such as, e.g., cardiopulmonary bypass (CPB) surgery, PCI and stent placement. Reference value for the ACT can range from between 70-180 seconds. However, for certain procedures such as CPB the desired range can exceed 400-500 seconds. ACT utilizes negatively charged particles for a determination of time to clot formation. Examples of various particles that can be used include celite, which has a normal length of ACT being about 100 to 170 seconds; kaolin, which has a normal length of ACT being about 90 to 150 seconds; and glass particles, which have a normal length of ACT being about 190 to 300 seconds. Suitable machines for measuring ACT include, e.g., Hemochron and Medtronic HemoTec.

In the aPTT (also referred to as “partial thromboplastin time” or “PTT”) test, a contact activator is used to stimulate the production of Factor XIIa by providing a surface for the function of high molecular weight kininogen, kallikrein and Factor XIIa. This contact activation is allowed to proceed for a specific period of time. Calcium is then added to trigger further reactions and the time required for clot formation is measured. Phospholipids are required to form complexes, which activate Factor X and Prothrombin. APTT can be measured by the IL TestTM APTT-SP(liquid). Reference values for aPTT are about 25 to 35 seconds. A prolonged aPTT indicates that clotting is taking longer than expected, e.g., due to a heparin or LMWH treatment.

### Methods of Making LMWH Preparations

Various methods of making LMWH preparations, e.g., a LMWH preparation described herein are provided in U.S. Publication No.: 2007/0287683, the contents of which is incorporated herein by reference.

M118-REH, as used herein, refers to the M118-REH composition described in U.S. Publication No.: 2007/0287683.

### Pharmaceutical Compositions

Compositions, e.g., pharmaceutically acceptable compositions, which include a LMWH preparation described herein, formulated together with a pharmaceutically acceptable carrier, are provided.

As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g., by injection or infusion).

The compositions of this invention may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical preferred compositions are in the form of injectable or infusible solutions. The preferred mode of administration is parenteral (e.g., intravenous, subcutaneous, intraperitoneal, intramuscular). In a preferred embodiment, the LMWH preparation is administered by intravenous infusion or injection. In another preferred embodiment, the LMWH preparation is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intravitreous, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high concentration. Sterile injectable solutions can be prepared by incorporating the active compound (i.e., LMWH in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, various polymers, monostearate salts and gelatin.

The LMWH preparations can be administered by a variety of methods known in the art, although for many therapeutic applications, the preferred route/mode of administration is intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Formulations for injection may be presented in unit dosage form, e.g., in ampoules or in multi-dose containers, e.g., with an added preservative. The compositions may take such forms as suspensions, solutions or emulsions in oily or aqueous vehicles, and may contain formulatory agents such as suspending, stabilizing and/or dispersing agents.

In addition to the compositions described previously, the compounds may also be formulated as a depot preparation. Such long-acting formulations may be formulated with suitable polymeric or hydrophobic materials (for example, as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions.

The pharmaceutical compositions of the invention may include a “therapeutically effective amount” or a “prophylactically effective amount” of a LMWH preparation. A “therapeutically effective amount” refers to an amount effective, at dosages and for periods of time necessary, to achieve a desired therapeutic result. A therapeutically effective amount of the LMWH preparation may vary according to factors such as the disease state, age, sex, and weight of the individual, and the LMWH preparation to elicit a desired response in the individual. A therapeutically effective amount is also one in which any toxic or detrimental effects of the LMWH preparation is outweighed by the therapeutically beneficial effects. A “therapeutically effective dosage” preferably inhibits a measurable parameter, e.g., coagulation or thrombosis, e.g., as measured by ACT and aPTT, by at least about 20%, more preferably by at least about 40%, even more preferably by at least about 60%, and still more preferably by at least about 80% relative to untreated subjects. The ability of a compound to inhibit a measurable parameter, e.g., coagulation or thrombosis, can be evaluated in an animal model system predictive of efficacy in humans. Alternatively, this property of a composition can be evaluated by examining the ability of the compound in an in vitro assay. Exemplary doses for intravenous administration of the LMWH preparation are about 1 IU/kg to 200 IU/kg, e.g., 1 IU/kg; 2 IU/kg; 3 IU/kg, 4 IU/kg, 5 IU/kg, 6 IU/kg, 7 IU/kg, 8 IU/kg, 9 IU/kg, 10 IU/kg, 11 IU/kg, 12 IU/kg, 13 IU/kg, 14 IU/kg, 15 IU/kg, 16 IU/kg, 17 IU/kg, 18 IU/kg, 19 IU/kg, 20 IU/kg, 21 IU/kg, 22 IU/kg, 25 IU/mg, 30N/kg, 40 IU/kg, 50N/kg, 70 IU/kg, 100 IU/kg, 125 IU/kg, 150N/kg, 175 IU/kg, 200 IU/kg. Other exemplary doses for intravenous administration of the LMWH preparation are about 0.03 mg/kg to 0.45 mg/kg, e.g., 0.03 mg/kg, 0.05 mg/kg, 0.1 mg/kg, 0.15 mg/kg, 0.2 mg/kg, 0.22 mg/kg, 0.25 mg/kg, 0.27 mg/kg, 0.3 mg/kg, 0.35 mg/kg, 0.37 mg/kg, 0.4 mg/kg, 0.44 mg/kg, preferably about 0.1 mg/kg, 0.15 mg/kg, 0.2 mg/kg, 0.25 mg/kg, 0.3 mg/kg, 0.35 mg/kg, 0.4 mg/kg, 0.44 mg/kg, 0.47 mg/kg, 0.5 mg/kg, 0.55 mg/kg, 0.60 mg/kg, 0.7 mg/kg, preferably about 0.30 to 0.50 mg/kg, e.g., 0.30 mg/kg, 0.35 mg/kg, 0.40 mg/kg, 0.42 mg/kg, 0.44 mg/kg, 0.47 mg/kg or 0.50 mg/kg.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the invention are kits comprising a LMWH preparation provided herein. The kit can include one or more other elements including: instructions for use in the absence of a GPIIb/IIIa inhibitor and/or for a subject having one or more characteristic described herein. The kit can also include other reagents, e.g., a therapeutic agent or protamine sulfate; devices or other materials for preparing the LMWH preparation for administration; pharmaceutically acceptable carriers; and devices or other materials for administration to a subject. Instructions for use can also include instructions for monitoring anti-Xa activity and/or anti-IIa activity using coagulation assays such as ACT and aPTT. The instructions can include instructions for therapeutic application including suggested dosages and/or modes of administration, e.g., in a patient having a disorder, e.g., a disorder described herein. Other instructions can include instructions on reversing anti-Xa activity and/or anti-IIa activity using protamine sulfate. The kit can further contain at least one additional reagent, such as a diagnostic or therapeutic agent, e.g., a diagnostic or therapeutic agent as described herein, formulated as appropriate, in one or more separate pharmaceutical preparations. The kit can include instructions regarding patients having a low platelet count (e.g., current platelet count or platelet count from a previous treatment with a GPIIb/IIIa inhibitor), patients having or who have had infusion site reaction (e.g., infusion site reaction from a previous treatment with a GPIIb/IIIa inhibitor), patients having antibodies to a GPIIb/IIIa inhibitor.

### Prophylactic and Therapeutic Uses

The LMWH preparations can be used to treat a subject. As used herein, the term “treat” or “treatment” is defined as the application or administration of a LMWH preparation to a subject, e.g., a patient, or application or administration to an isolated tissue or cell from a subject, e.g., a patient, which is returned to the patient. The subject can be a patient having a disorder (e.g., a disorder as described herein), a symptom of a disorder or a predisposition toward a disorder. The treatment can be to cure, heal, alleviate, relieve, alter, remedy, ameliorate, palliate, improve or affect the disorder, the symptoms of the disorder or the predisposition toward the disorder. As used herein, a subject is a vertebrate such as a human, non-human primate, cow, horse, pig, sheep, goat, dog, cat, or rodent. The subject can be, e.g., an experimental animal, a veterinary animal, or a human subject. A treatment can be therapeutic, e.g., a treatment which cures, heals, alleviates, relieves, alters, remedies, ameliorates, palliates, improves or affects the disorder or a symptom of the disorder, e.g., lessens, mitigates or ameliorates an existing unwanted condition or symptom thereof, or prophylactic, e.g., a treatment which delays, e.g., prevents, the onset of an unwanted condition or symptom thereof.

Heparins and LMWHs have many therapeutic utilities. The LMWH preparations provided herein can be used for the treatment of any type of condition in which heparin or LMWH therapy is useful. Thus, the preparations and methods are useful in a variety of in vitro, in vivo and ex vivo methods. For instance, it is known that heparins and LMWHs are useful for preventing and treating thrombotic disorders (e.g., ACS, stable or unstable angina, MI (e.g., STEMI and NSTEMI)) or cardiovascular disease (atherosclerosis), vascular conditions or arterial fibrillation. Disorders suitable for treatment with a heparin or LMWH are well-known in the art and is described, for instance, in Harrison's Principles of Internal Medicine (McGraw Hill, Inc., New York), which is incorporated by reference. The use of HLGAG compositions in various therapeutic methods is described and summarized in Huang, J. and Shimamura, A., Coagulation Disorders, 12, 1251-1281 (1998).

Thus, the LMWH preparations are useful for treating or preventing disorders associated with coagulation. When an imbalance in the coagulation pathway shifts towards excessive coagulation, the result is the development of thrombotic tendencies, which are often manifested as heart attacks, strokes, DVT, ACS, stable and unstable angina, and myocardial infarcts. A “disease associated with coagulation” as used herein refers to a condition characterized by local inflammation which can result from an interruption or reduction in the blood supply to a tissue which may occur, for instance, as a result of blockage of a blood vessel responsible for supplying blood to the tissue such as is seen for myocardial or cerebral infarction or peripheral vascular disease, or as a result of emboli formation associated with conditions such as arterial fibrillation, DVT or PE. Persons undergoing surgery, anesthesia and extended periods of bed rest or other inactivity are often susceptible to a condition known as deep venous thrombosis, or DVT, which is a clotting of venous blood in the lower extremities and/or pelvis. This clotting occurs due to the absence of muscular activity in the lower extremities required to pump the venous blood (stasis), local vascular injury or a hypercoaguble state. The condition can be life-threatening if a blood clot migrates to the lung, resulting in a “pulmonary embolus” or otherwise interferes with cardiovascular circulation. One method of treatment involves administration of an anti-coagulant.

The methods are useful for treating thrombotic disorders and cardiovascular disease. Cardiovascular disease includes, but is not limited to, atherosclerosis and arterial fibrillation. Atrial fibrillation is a common form of arrhythmia generally arising as a result of emotional stress or following surgery, exercise, or acute alcoholic intoxication. Arterial fibrillation is characterized by disorganized arterial activity without discrete P waves on the surface ECG. This disorganized activity can lead to improper blood flow in the atrium and thrombus formation. These thrombi can embolize, resulting in cerebral ischemia and other disorders.

Thrombotic disorders include, but are not limited to, ACS, e.g., MI, stable and unstable angina. Myocardial infarction is a disease state which sometimes occurs with an abrupt decrease in coronary blood flow that follows a thrombotic occlusion of a coronary artery previously narrowed by atherosclerosis. Such injury may be produced or facilitated by factors such as cigarette smoking, hypertension, and lipid accumulation. Angina is due to transient myocardial ischemia. This disorder is usually associated with a heaviness, pressure, squeezing, smothering, or choking feeling below the sternum. Episodes are usually caused by exertion or emotion, but can occur at rest. STEMI, also referred to as “Q wave myocardial infarction”, refers to MI with an abnormal echocardiogram. NSTEMI, or “non-Q wave myocardial infarction”, is not associated an echocardiogram abnormality. Stable angina occurs at predictable times with a specific amount of exertion or activity. Unstable angina may occur as a change in the usual pattern of stable angina. It my include chest pain that occurs at rest or with less and less exertion, that may be more severe and last longer, or that is less responsive to nitroglycerin. Unstable angina means that blood flow has gotten worse potentially by an increased narrowing or small blood clots that form in the coronary arteries. Unstable angina is a warning sign that myocardial infarction may soon occur.

A “major bleeding event” as used herein refers to intracranial hemorrhage and other bleeding events that decrease hemoglobin levels greater than 5 g/dL. A “minor bleeding event” as used herein refers to bleeding that decrease hemoglobulin levels greater than 3 g/dL, or unidentified bleeding that decrease hemoglobulin levels greater than 4 g/dL.

The LMWH preparation can be used for the treatment of thrombotic and cardiovascular disorders alone or in combination with other therapeutic agents for reducing the risk of a cardiovascular disease or for treating the cardiovascular disease. For example, the combination therapy can include a LMWH preparation coformulated with, and/or coadministered with, one or more additional therapeutic agents, e.g., one or more therapeutic agent described herein. Administered “in combination”, as used herein, means that two (or more) different treatments are delivered to the subject during the course of the subject's affliction with the disorder, e.g., the two or more treatments are delivered after the subject has been diagnosed with the disorder or identified as at risk for the disorder and before the disorder has been prevented, cured or eliminated. In some embodiments, the delivery of one treatment is still occurring when the delivery of the second begins, so that there is overlap. This is sometimes referred to herein as “simultaneous” or “concurrent delivery.” In other embodiments, the delivery of one treatment ends before the delivery of the other treatment begins. The delivery can be such that an effect of the first treatment delivered is still detectable when the second is delivered. Other therapeutic agents include, but are not limited to, anti-inflammatory agents, anti-thrombotic agents, anti-platelet agents, fibrinolytic agents, thrombolytics, lipid reducing agents, direct thrombin inhibitors, anti-Xa inhibitors, anti-IIa inhibitors, and direct thrombin inhibitors. Examples of agents that can be administered in combination with the LMWH preparations provided herein include bivalirudin, hirudin, hirugen, Angiomax, agatroban, PPACK, thrombin aptamers, aspirin, P2Y12 inhibitors, thienopyridine, ticlopidine, and clopidogrel.

The monitorability by standard anticoagulation assays such as ACT and aPTT as well as the reversibility of the LMWH preparations provided herein provided improved flexibility in treating patients such as those patients admitted to the hospital and undergoing evaluation for possible cardiovascular surgery. Such benefits are highlighted by the following scenario. A patient goes the hospital complaining of symptoms that can be associated with various thrombotic disorders such as ACS including stable angina, unstable angina and MI. The monitorability and reversibility of the LMWH preparations provided herein allow use of such preparations while the patient is being evaluated for potential cardiovascular surgery. If it is determined that the patient will receive surgical intervention such as PCI or stent placement, the monitorability of the LMWH preparations, the anti-Xa activity and anti-IIa activity of the LMWH preparation can be monitored during the procedure, and, if necessary, one or more additional doses of the LMWH preparation can be given during or after the procedure to maintain these activities. If it is determined that a patient will receive a surgical intervention such as CABG, the anti-Xa activity and anti-IIa activity of the LMWH preparation can be neutralized with protamine sulfate prior to surgical intervention. In addition, anti-Xa activity and anti-IIa activity can be monitored in the patient to ensure the activity is sufficiently decreased prior to the surgery.

The LMWH preparations provided herein are also useful for treating vascular conditions. Vascular conditions include, but are not limited to, disorders such as DVT, peripheral vascular disease, cerebral ischemia, including stroke, and PE. A cerebral ischemic attack or cerebral ischemia is a form of ischemic condition in which the blood supply to the brain is blocked. This interruption or reduction in the blood supply to the brain may result from a variety of causes, including an intrinsic blockage or occlusion of the blood vessel itself, a remotely originated source of occlusion, decreased perfusion pressure or increased blood viscosity resulting in inadequate cerebral blood flow, or a ruptured blood vessel in the subarachnoid space or intracerebral tissue. The methods are useful for treating cerebral ischemia. Cerebral ischemia may result in either transient or permanent deficits and the seriousness of the neurological damage in a patient who has experienced cerebral ischemia depends on the intensity and duration of the ischemic event. A transient ischemic attack is one in which the blood flow to the brain is interrupted only briefly and causes temporary neurological deficits, which often are clear in less than 24 hours. Symptoms of TIA include numbness or weakness of face or limbs, loss of the ability to speak clearly and/or to understand the speech of others, a loss of vision or dimness of vision, and a feeling of dizziness. Permanent cerebral ischemic attacks, also called stroke, are caused by a longer interruption or reduction in blood flow to the brain resulting from either a thrombus or embolism. A stroke causes a loss of neurons typically resulting in a neurologic deficit that may improve but that does not entirely resolve.

Thromboembolic stroke is due to the occlusion of an extracranial or intracranial blood vessel by a thrombus or embolus. Because it is often difficult to discern whether a stroke is caused by a thrombosis or an embolism, the term “thromboembolism” is used to cover strokes caused by either of these mechanisms.

The methods are also directed to the treatment of acute thromboembolic stroke using a LMWH preparation provided herein. An acute stroke is a medical syndrome involving neurological injury resulting from an ischemic event, which is an interruption or reduction in the blood supply to the brain.

An effective amount of a LMWH preparation alone or in combination with another therapeutic for the treatment of stroke is that amount sufficient to reduce in vivo brain injury resulting from the stroke. A reduction of brain injury is any prevention of injury to the brain which otherwise would have occurred in a subject experiencing a thromboembolic stroke absent the treatment described herein. Several physiological parameters may be used to assess reduction of brain injury, including smaller infarct size, improved regional cerebral blood flow, and decreased intracranial pressure, for example, as compared to pretreatment patient parameters, untreated stroke patients or stroke patients treated with thrombolytic agents alone.

The LMWH preparation may be used alone or in combination with a therapeutic agent for treating a disease associated with coagulation. Examples of therapeutics useful in the treatment of diseases associated with coagulation include anticoagulation agents, antiplatelet agents, and thrombolytic agents.

Anticoagulation agents prevent the coagulation of blood components and thus prevent clot formation. Anticoagulants include, but are not limited to, warfarin, Coumadin, dicumarol, phenprocoumon, acenocoumarol, ethyl biscoumacetate, and indandione derivatives. “Direct thrombin inhibitors” include hirudin, hirugen, Angiomax, agatroban, PPACK, thrombin aptamers. Antiplatelet agents inhibit platelet aggregation and are often used to prevent thromboembolic stroke in patients who have experienced a transient ischemic attack or stroke. Thrombolytic agents lyse clots which cause the thromboembolic stroke. Thrombolytic agents have been used in the treatment of acute venous thromboembolism and pulmonary emboli and are well known in the art (e.g. see Hennekens et al, J Am Coll Cardiol; v. 25 (7 supp), p. 18S-22S (1995); Holmes, et al, J Am Coll Cardiol; v. 25 (7 suppl), p. 10S-17S(1995)).

Pulmonary embolism as used herein refers to a disorder associated with the entrapment of a blood clot in the lumen of a pulmonary artery, causing severe respiratory dysfunction. Pulmonary emboli often originate in the veins of the lower extremities where clots form in the deep leg veins and then travel to lungs via the venous circulation. Thus, pulmonary embolism often arises as a complication of deep venous thrombosis in the lower extremity veins. Symptoms of pulmonary embolism include acute onset of shortness of breath, chest pain (worse with breathing), and rapid heart rate and respiratory rate. Some individuals may experience haemoptysis.

The preparations and methods are also useful for treating or preventing atherosclerosis. Heparin has been shown to be beneficial in prevention of atherosclerosis in various experimental models. Atherosclerosis is one form of arteriosclerosis that is believed to be the cause of most coronary artery disease, aortic aneurysm and atrial disease of the lower extremities, as well as contributing to cerebrovascular disease.

The LMWH preparations are also useful before, during or after surgical and dialysis procedures. Surgical patients, especially those over the age of 40 years have an increased risk of developing DVT. Thus, the use of the LMWH preparations provided herein for preventing the development of thrombosis associated with surgical procedures is contemplated. In addition to general surgical procedures such as percutaneous intervention (e.g., percutaneous coronary intervention (PCI)), PCTA, stents and other similar approaches, hip or knee replacement, cardiac-pulmonary by-pass surgery, coronary revascularization surgery, orthopedic surgery, and prosthesis replacement surgery, the methods are also useful in subjects undergoing a tissue or organ transplantation procedure or treatment for fractures such as hip fractures.

The treatments provided herein can further include administering protamine sulfate to neutralize the anti-Xa activity and/or anti-IIa activity of the LMWH preparation, e.g., once anti-coagulation or anti-thrombotic activity is no longer necessary. Protamine sulfate can be administered, e.g., by intravenous administration, at a dose of about 1, 2, 3 mg of protamine sulfate per 100 IU of anti-Xa activity. The IUs of anti-Xa activity can be determined using, e.g., the coagulation assays described herein.

### Other Embodiments

This invention is further illustrated by the following examples that should not be construed as limiting. The contents of all references, patents and published patent applications cited throughout this application are incorporated herein by reference.

### EXAMPLES

### Example 1

### Eminence Study Design

The Eminence study is a randomized, open-label, active controlled, dose-ranging trial in patients undergoing elective PCI. Pre-PCI antiplatelet (ASA, clopidogrel) therapy was used but planned use of GP IIb/IIIa was not allowed. Approximately 500 patients with stable coronary artery disease undergoing elective PCI were randomly assigned to receive treatment with one of three doses of intravenous M118-REH or a standard dose of unfractionated heparin (UFH).

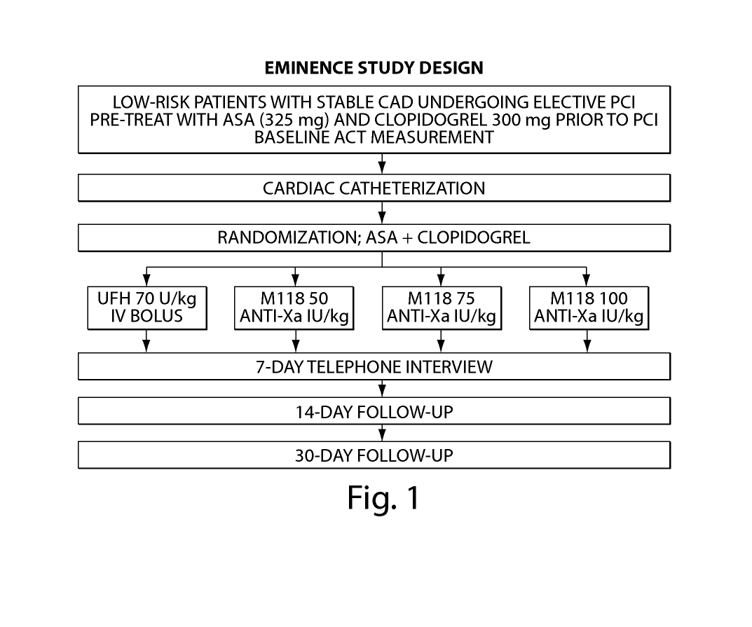

The trial had 4 arms: 70 U/kg UFH, 50 IU/kg M118, 75 IU/kg M118, 100 IU/kg M118-REH. The primary endpoint of the study was the combined incidence of clinical events defined as the composite of death, myocardial infarction, repeat revascularization, and stroke (over thirty days); incidence of bleeding and thrombocytopenia (over the first 24 hours); and bailout use of glycoprotein IIb/IIIa inhibitors and catheter thrombus (during the procedure). A schematic of the trial design is shown in FIG. 1.

### Example 2

### Trial Results

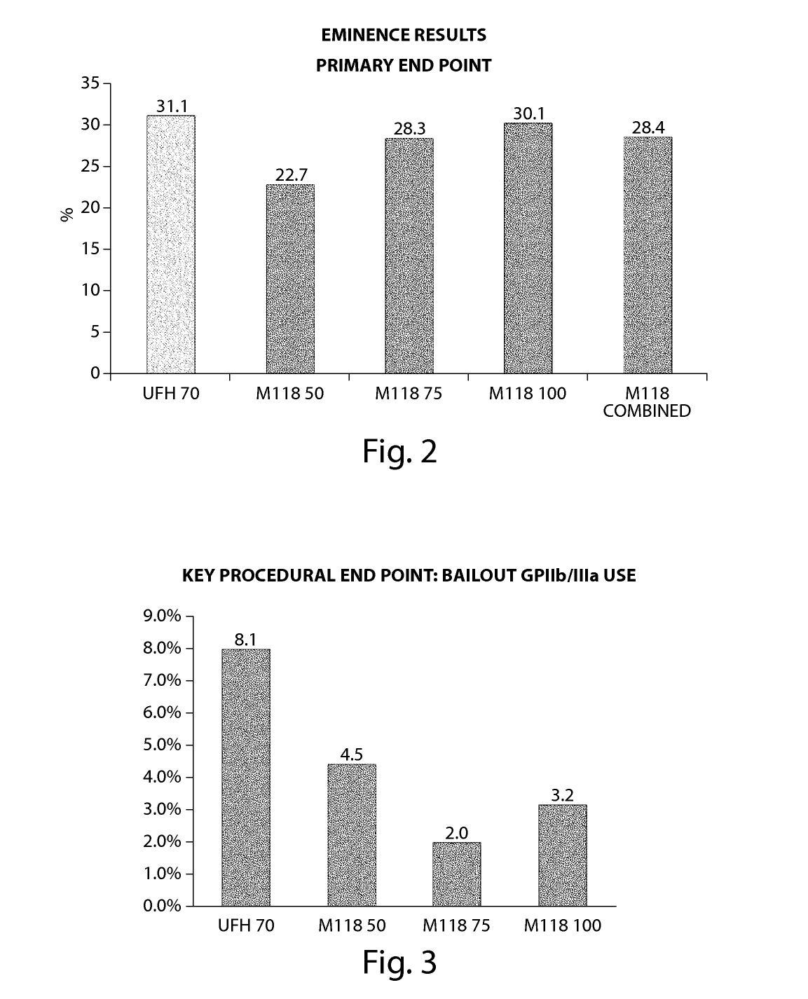

The analysis of the study provided evidence of non-inferiority of the combined M118-REH group (combining all three doses) as compared to the UFH group within the parameters of the prospectively defined endpoints. The observed incidence of the composite endpoint was lower in all M118-REH treatment groups than in the UFH group; however it should be noted that the study was not designed or powered to detect statistically significant differences in the primary endpoint between treatments. The incidence of serious and non-serious adverse events was comparable in all treatment groups (FIG. 2).

Unexpectedly, however, significant differences were seen in a specific key procedural endpoint: bailout glycoprotein (GP) IIb/IIIa utilization. GP IIb/IIIa inhibitors are often used as a rescue or bailout therapy to manage complications arising during percutaneous coronary intervention. As shown in FIG. 3, it was unexpectedly discovered that M118-REH demonstrated lower bailout GPIIb/IIIa utilization than the comparator UFH at all dosages of M118-REH tested. As GP IIb/IIIa inhibition inhibits the function of platelets and can to lead to an increase in bleeding, the risk of bleeding may be reduced for patients administered M118-REH as less bailout use of GPIIb/IIIa is required. Accordingly, M118-REH may be an important choice for physicians treating patients who need an anticoagulant, but are at risk of increased bleeding or at risk for a negative reaction to a GPIIb/IIIa inhibitor.

1. A method of reducing the risk of a major or minor bleeding event in a subject being treated for a thrombotic or cardiovascular disorder, the method comprising:
administering a LMWH composition having
a weight average molecular weight of about 5000 to 9000 Da; and
an anti-IIa activity of about 100 to 300 IU/mg;
to a subject having or at risk for having a thrombotic or cardiovascular disorder in the absence of treatment with a GPIIb/IIIa inhibitor, to thereby reduce the risk of a major or minor bleeding event in the subject.

2. The method of claim 1, wherein the thrombotic disorder is an acute coronary syndrome.

3. (canceled)

4. A method for treating a subject having or at risk of having a thrombotic or cardiovascular disorder, the method comprising:
selecting a subject for treatment with a LMWH composition having
a weight average molecular weight of about 5000 to 9000 Da; and
an anti-IIa activity of about 100 to 300 IU/mg;
in the absence of a GPIIb/IIIa inhibitor, on the basis that the subject has a predisposition for bleeding; and
administering a LMWH composition described herein, e.g., in the absence of treatment with a GPIIb/IIIa inhibitor, to the subject.

5. The method of claim 4, further comprising administering the LMWH composition to the subject at a dose between about 60 IU/kg and about 150 IU/kg.

6. The method of claim 5, wherein the LMWH composition is administered subcutaneously or by intravenous (i.v.) infusion.

7. The method of claim 5, wherein the thrombotic disorder is an acute coronary syndrome.

8. The method of claim 5, wherein treatment may require surgical intervention such as PCI, stent placement or atherectomy.

9. A method of treating a subject having or at risk of having a thrombotic disorder or a cardiovascular disorder for treatment with a LMWH composition having
a weight average molecular weight of about 5000 to 9000 Da; and
an anti-IIa activity of about 100 to 300 IU/mg;
the method comprising
identifying a subject having or at risk of having a thrombolytic disorder and that has a platelet count less than a standard or that has had a platelet count less than a standard from a previous treatment that comprised a GPIIb/IIIa inhibitor; and
determining if the subject as suitable for treatment with the LMWH composition in the absence of treatment with a GPIIb/IIIa inhibitor; and
administering the LMWH composition to the subject.

10.-16. (canceled)

17. A method of treating a subject having or at risk of having a thrombotic or cardiovascular disorder, the method comprising
selecting a subject having or at risk of having a thrombotic or cardiovascular disorder that has had a platelet count less than a standard, or that has a platelet count less than a standard, and
administering a LMWH composition having
a weight average molecular weight of about 5000 to 9000 Da; and
an anti-IIa activity of about 100 to 300 IU/mg;
in the absence of treatment with a GPIIb/IIIa inhibitor, to the subject.

18.-25. (canceled)

26. A method for treating a subject having or at risk of having a thrombotic or cardiovascular disorder, the method comprising:
selecting a subject for treatment with a LMWH composition having
a weight average molecular weight of about 5000 to 9000 Da; and
an anti-IIa activity of about 100 to 300 IU/mg;
on the basis that the subject has creatinine clearance levels less than a standard; and
administering the LMWH composition in the absence of a GPIIb/IIIa inhibitor to the subject.

27.-36. (canceled)

37. A method for treating a subject having or at risk of having a thrombotic or cardiovascular disorder, the method comprising:
selecting a subject for treatment with a LMWH composition having
a weight average molecular weight of about 5000 to 9000 Da; and
an anti-IIa activity of about 100 to 300 IU/mg; on the basis that the subject has a systolic blood pressure levels and/or a diastolic blood pressure level greater than a standard; and
administering the LMWH composition in the absence of a GPIIb/IIIa inhibitor, to the subject.

38.-41. (canceled)

42. A method for treating a subject having or at risk of having a thrombotic or cardiovascular disorder, comprising:
selecting a subject for treatment with a LMWH composition having
a weight average molecular weight of about 5000 to 9000 Da; and
an anti-IIa activity of about 100 to 300 IU/mg; on the basis that the subject receives dialysis treatment, and
administering the LMWH composition in the absence of a GPIIb/IIIa inhibitor, to the subject.

43.-53. (canceled)

54. A method for treating a subject having or at risk of having a thrombotic or cardiovascular disorder, the method comprising:
selecting a subject on the basis that the subject has tested positive for the production of antibodies to treatment with a GPIIb/IIIa inhibitor, or on the basis that the subject is at risk for developing antibodies to treatment with a GPIIb/IIIa inhibitor; and
administering a LMWH composition having
a weight average molecular weight of about 5000 to 9000 Da; and
an anti-IIa activity of about 100 to 300 IU/mg;
in the absence of a GPIIb/IIIa inhibitor, to the subject.

55.-63. (canceled)

64. A method of treating a subject having or at risk of having a thrombotic or cardiovascular disorder, the method comprising:
selecting a subject for treatment with a LMWH composition composition having
a weight average molecular weight of about 5000 to 9000 Da; and
an anti-IIa activity of about 100 to 300 IU/mg;
on the basis that the subject is in need of reduced infusion site reaction; and
administering the LMWH to the subject in the absence of a GPIIb/IIIa inhibitor.

65.-68. (canceled)

69. A method for evaluating a subject having or at risk of having a thrombotic or cardiovascular disorder, the method comprises:
evaluating one or more of the following parameters of the subject:
platelet count, creatinine clearance levels, infusion site reaction, presence of antibodies to a GPIIb/IIIa inhibitor, or blood pressure; and
based on the evaluation making a decision about treatment with unfractionated heparin or a LMWH composition having
a weight average molecular weight of about 5000 to 9000 Da; and
an anti-IIa activity of about 100 to 300 IU/mg;
for the subject.

70.-80. (canceled)

81. The method of claim 1 further comprising:
determining if the subject having or at risk of having a thrombotic or cardiovascular disorder has a predisposition to bleeding.

82. The method of claim 81, wherein the subject has exhibited one or more bleeding event within the past 30 days.

83. The method of claim 7, wherein the thrombotic disorder unstable angina, non-ST segment elevated myocardial infarction (NSTEMI) or a ST segment elevated myocardial infarction (STEMI).
